(19) United States
(12) Patent Application Publication
(10) Pub. No.: US 2010/0087534 A1
(43) Pub. Date: Apr. 8, 2010

(54) Use of a Photosensitizing Agent in the Treatment or Prevention of an Inflammation-Associated Disorder in the Gastrointestinal Tract of a Mammal

(76) Inventors: Maria-Anna Ortner, Glion (CH); Pierre Michetti, Lausanne (CH); Dominique Velin, Sergy (FR)

Correspondence Address: HARNESS, DICKEY & PIERCE, P.L.C., P.O. BOX 8910, RESTON, VA, 20195, US

(21) Appl. No.: 11/992,963

(22) PCT Filed: Jul. 5, 2006
(86) PCT No.: PCT/IB2006/001857

§ 371 Date: Dec. 9, 2009

(30) Foreign Application Priority Data
Jul. 5, 2005 (IB) PCT/IB2005/001900

(51) Int. Cl. A61K 31/195; C07C 229/04; A61P 29/00; A61P 1/00

(52) U.S. Cl. 514/561; 562/567

(57) ABSTRACT

The present invention relates generally to the field of Photodynamic therapy (PDT) and more particularly to the use of a photosensitizing agent for the preparation of a medicament for the treatment or prevention of an inflammation-associated disorder in the gastrointestinal tract of a mammal, wherein the expression of pro-inflammatory markers in a tissue of said gastrointestinal tract is decreased after administering said photosensitizing agent to said tissue and exposing said tissue to a light having a wavelength absorbed by said photosensitizing agent.

### FIELD OF THE INVENTION

The present invention relates generally to the field of Photodynamic therapy (PDT) and more particularly to the use of a photosensitizing agent for the preparation of a medicament for the treatment or prevention of an inflammation-associated disorder in the gastrointestinal tract of a mammal, wherein the expression of pro-inflammatory markers in a tissue of said gastrointestinal tract is decreased after administering said photosensitizing agent to said tissue and exposing said tissue to a light having a wavelength absorbed by said photosensitizing agent.

### BACKGROUND OF THE INVENTION

Photodynamic therapy (PDT) uses the photo-physical properties of naturally occurring or synthetically derived light-absorbing molecules (photosensitizing agents or photosenzitizer) that efficiently generate reactive oxygen species upon exposure to light. The general method of performing PDT is now well known and described, for example, in U.S. Pat. Nos. 4,968,715; 4,932,934; and 5,028,621 (Dougherty et al.) and U.S. Pat. No. 5,002,962 (Pandea et al.). Administration of a photosensitizer, is followed by activation of the drug with non-thermal light of a specific wavelength (Dougherty et al. Photodynamic therapy. J Natl Cancer Inst 1998). The interaction of light with a photosensitizer molecule raises its energy state in the presence of molecular oxygen. This leads to the formation of reactive oxygen species, primarily singlet oxygen (¹O₂). Following light irradiation, PDT rapidly induces apoptosis in a wide variety of cell types in vitro.

For cancer indications, PDT is typically given as a localized intense treatment that leads to tumor killing most likely through a direct effect of these oxygen species against tumor cells, as well as an antivascular action that impairs blood supply to the region. The exact mechanism, however, is still unknown.

Non-cancer indications responsive to PDT now include ocular (age-related macular degeneration) and cardiovascular (restenosis) disorders.

Some work has been done with PDT to achieve an anti-inflammatory effect, in particular in inflammation arising from injured ocular tissue following either glaucoma filtering surgery (see International Patent Application WO98/34644, Stewart et al.) or after treatment from normal dose PDT (see International Patent Application WO02/064163, Margaron et al.). However, as can be seen from these two International Patent Applications, the effect of PDT on inflammation might be positive or negative depending on the photosensitizing agent and the light dose applied as well depending on the tissue treated.

There has been some progress in the treatment of inflammation-associated disorders in the gastrointestinal tract with biological therapies including the anti-TNFα-antibody Infliximab®, but the effect of a single dose is short-lived, repeated dosing can induce serious side effects and long-term safety of this medication is not established. Therefore, it is an object of the present invention to provide new treatment modalities for the treatment of inflammation-associated disorders in the gastrointestinal tract which have a good safety profile, only low or no side effects and the possibility to retreat, whenever necessary.

This object has been achieved by providing the use of a photosensitizing agent for the preparation of a medicament for the treatment or prevention of an inflammation-associated disorder in the gastrointestinal tract of a mammal, wherein the expression of pro-inflammatory markers in a tissue of said gastrointestinal tract is decreased after administering said photosensitizing agent to said tissue and exposing said tissue to a light having a wavelength absorbed by said photosensitizing agent.

### SUMMARY OF THE INVENTION

The present invention concerns the use of a photosensitizing agent for the preparation of a medicament for the treatment or prevention of an inflammation-associated disorder in the gastrointestinal tract of a mammal, wherein the expression of pro-inflammatory markers in a tissue of said gastrointestinal tract is decreased after administering said photosensitizing agent to said tissue and exposing said tissue to a light having a wavelength absorbed by said photosensitizing agent.

A further object of the present invention is the use of a photosensitizing agent for the preparation of a medicament for decreasing the expression of pro-inflammatory markers in the tissue of the gastrointestinal tract of a mammal having an inflammation-associated disorder of said gastrointestinal tract.

### BRIEF DESCRIPTION OF THE FIGURES

FIG. 1 show the expression index of the pro-inflammatory markers IFN-γ (A), IL-1Ra (B) and TNF-α (C). Y-axis: all data are given as expression index (group mean/mean in naïve mice), x-axis: groups are indicated.

FIG. 2 represents the expression indices of the proinflammatory markers IFN-γ and TNF-α in untransfered mice, disease control group and PDT group (15 mg/kg of δ-ALA, 10 J/cm²).

FIG. 3 shows the correlation between endoscopic severity index and proinflammatory markers IFN-γ and TNF-α expression.

FIG. 4: FIG. 4 A depicts the evolution of endoscopic severity index of marked inflamed CD4⁺ CD45RB^{high} transferred SCID mice treated by low dose PDT (15 mg/kg δ-ALA and 10 J/cm² illumination energy) compared to the non-treated disease control group (DC) and unmanipulated (UM) mice. FIG. 4 B shows examples of colonoscopic pictures demonstrating the improvement of the endoscopic appearance of treated colons. Left panels: pre-PDT status of the colons of marked inflamed mice displaying masked vascular patterns, granularity and presence of small ulcers (arrows). Right panels: same portions of the colons of the same mice observed 3 days post PDT implementation. Vascular patterns were unmasked, granularity has disappeared and ulcers appeared to be resorbed in PDT-treated (lower panel). No modification was observed in DC mice (upper panel). Some mice were sacrificed 3 days post PDT. UM mice were also sacrificed but at the end of the colonoscopic monitoring trial. mRNA were extracted for the collected colons and were subjected to reverse transcription. FIG. 4 C depicts the expression index of these mRNA coding for IL-17 and IL-6 (C, two right panels) based on real-time PCR analyses. Bars represent mean values±SEMs. Significant statistical differences are indicated. *:p<0.05; **:p<0.01; ***:p<0.0001.

FIG. 5: FIG. 5 A shows the evolution of the colitis activity of moderately inflamed CD4⁺ CD45RB^{high} transferred SCID mice after low dose PDT (15 mg/kg δ-ALA and 10 J/cm² illumination energy) compared to the disease control group (DC) and unmanipulated (UM) SCID mice as negative control. FIG. 5 B represents two groups of marked inflamed CD4⁺ CD45RB^{high} transferred SCID mice were treated by low dose PDT (15 mg/kg δ-ALA) with either illumination energy of 20 J/cm² or of 2 J/cm². Evolution of the colitis activity was colonoscopically monitored at 3 days, 1, 2, 3 and 4 weeks post PDT implementation. As points of comparison, evolution of the previously exploited PDT regimen consisting of an illumination energy of 10 J/cm² (see FIG. 4 A) as well as of DC and UM mice of related experiments, are also illustrated on this graph. FIG. 5 C represents marked inflamed CD4⁺ CD45RB^{high} transferred SCID mice treated by low dose PDT (15 mg/kg δ-ALA and 10 J/cm² illumination energy). Evolution of the colitis activity was colonoscopically monitored at 3 days and 1 week post PDT implementation; age-matched, non-transferred, unmanipulated (UM) SCID mice served as negative control. Chart of significant statistical differences for all graphs; *:p<0.05; **:p<0.01.

FIG. 6 shows that low dose PDT treatment induces diminution in the number of CDe cells in the mucosa of treated colons 3 days after PDT implementation. Marked inflamed mice were treated by low dose PDT (15 mg/kg δ-ALA and 10 J/cm² illumination energy) and were sacrificed either 4 or 20 hours after PDT implementation. The percentage of Annexin r cells within CD4⁺ cells was analyzed in a forward and side scatter gated cell population consisting of viable cells. Bars represent mean values±SEMs. Significant statistical differences are indicated. **:p<0.01.

Usually the photosensitizing agent will be selected from the group comprising porphyrins, 5-aminolevulinic acid, benzoporphyrin-derivative mono acid-A, chlorins, purpurins, pheophorbides, pyropheophorbides, pheophytins, phorbins, phtalocyanines, naphthalocyanines, phenothiazine, methylene blue, texaphyrins, porphycenes, sapphyrins, synthetic dyes, hypericin.

Examplary porphyrins include hematoporphyrin, hematoporphyrin derivate (Photofrin®), verteporfin (Visudyne®), tetraphenylporphyrin and methoxyphenylporphyrin.

Examplary chlorins include meso-tetrahydroxyphenyl chlorin (Foscan®) and bateriochlorins.

Examplary synthetic dyes include xanthene dyes, toluidine blue, Rose Bengal, eosin, indigo carmine and indocyanine green.

Examplary purpurins include tin ethyl etiopurpurin (Purlytin®), octaethylpurpurin, octaethylpurpurin zinc, oxidized octaethylpurpurin, reduced octaethylpurpurin, reduced octaethylpurpurin tin, purpurin 18, purpurin-18, purpurin-18-methyl ester, purpurin, Zn (II) aetiopurpurin ethyl ester, and zinc etiopurpurin.

Preferably, the photosensitizing agent is 5-aminolevulinic acid (δ-ALA) or verteporfin.

In case the photosensitizing agent is a polypeptide, then the present invention also considers modified photosensitizing agent as long as it exhibits the same properties as the native sequence.

For example the photosensitizing agent may be prepared in order to include D-forms and/or “retro-inverso isomers” of the peptide(s). By “retro-inverso isomer” is meant an isomer of a linear peptide in which the direction of the sequence is reversed and the chirality of each amino acid residue is inverted; thus, there can be no end-group complementarity.

Protecting the peptide from natural proteolysis or chemical derivitization could increase the effectiveness of the specific heterobivalent or heteromultivalent photosensitizing agent.

Exemplary of polypeptidic photosensitizing agents are tyrosine and tryptosan photosensitized by a chiral pi,pi aromatic ketone, peptide-nucleic acids, Ala-Pro-Arg-Pro-Gly (APRPG) pentapeptide and PEG modified liposomal benzoporphyrin derivate monoacid ring A (APRPG-PEG-Lip BPD-MA).

Typically, the photosensitizing agent can be formulated for the preparation of a medicament by mixing the photosensitizing agent, typically at ambient temperatures, appropriate pH's, and the desired degree of purity, with one or more physiologically acceptable carriers, excipients, or stabilizers, i.e., that are non-toxic to recipients at the dosages and concentrations employed.

Preferably, suitable forms are powder, aqueous solvent mixtures, lipase-based formulations or liposome formulations.

Acceptable carriers, excipients, or stabilizers include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN®, PLURONICS® or polyethylene glycol (PEG).

The form of administration of the medicament may be systemic or topical. For example, administration of such a composition may be various parenteral routes such as subcutaneous, intravenous, intradermal, intramuscular, intraperitoneal, transdermal, oral routes or via an implanted device, and may also be delivered by peristaltic means.

Preferred administrations are topical, oral or intravenous.

The medicament comprising a photosensitizing agent, as described herein, as an active agent may also be incorporated or impregnated into a bioabsorbable matrix, with the matrix being administered in the form of a suspension of matrix, a gel or a solid support. In addition the matrix may be comprised of a biopolymer.

Sustained-release preparations may be prepared. Suitable examples of sustained-release preparations include semi permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides, copolymers of L-glutamic acid and [gamma]ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The medicaments to be used for in vivo administration must be sterile. This is readily accomplished for example by filtration through sterile filtration membranes.

“Inflammation-associated disorder” refers to a disease caused by an “inflammation”. “Inflammation” means changes that occur in a living body following an injury. The injury may be caused by physical agents, such as excessive heat or cold, pressure, ultraviolet or ionizing irradiation, cuts or abrasions; by a wide variety of inorganic or organic chemical substances; or by biological agents such as viruses, bacteria, and other parasites.

Typically, the inflammation-associated disorder in the gastrointestinal tract is selected from the group comprising, but not limited to, Crohn's disease, inflammatory bowel disease, microscopic colitis, autoimmune cholangiopathy, autoimmune pancreatitis, sarcoidosis, lupus erythematosus, sprue such as tropical and celiac disease, Whipple's disease, bacterial cholangitis, microscopic lymphocytic colitis, microscopic collagenous colitis, radiation colitis, AIDS manifestation in the gastrointestinal tract, eosiniophile gastroenteritis or esophagitis (Kagnoff M f Immunology and inflammation of the gastrointestinal tract in Slesenger and Fordtran, Fith Edition, Gastrointestinal Disease, Saunders Philadelphia, London, Toronto, Montreal, Sydney, Tokyo 1993).

Preferably, the inflammation-associated disorder in the gastrointestinal tract is Crohn's disease, inflammatory bowel disease, microscopic colitis, microscopic lymphocytic colitis, microscopic collagenous colitis or radiation colitis.

In some inflammatory diseases of the gastrointestinal tract, like inflammatory bowel disease or sclerosing cholangitis, inflammation is thought to result from an overwhelming and ongoing activation of the mucosal immune system, induced by antigens in genetically susceptible individuals under special environmental conditions. (Fiocchi Inflammatory bowel disease: etiology and pathogenesis: Gastroenterology 1998; Podolsky Inflammatory bowel disease. N Engl J Med 2002; Lee et al. Primary sclerosing cholangitis. N Engl J Med 1995). Discussed putative antigens which elicit the aberrant immune response are bacterial antigens of the normal luminal flora, luminal alimentary continents or toxic bile products and viral infections.

In inflammatory bowel disease it seems that the immune system responds incorrectly to the microenvironment in the lumen. It remains unclear whether this is primarily facilitated by a defect in the epithelial mucosal barrier function or by a disturbance of the mucosal immune system or by both factors. Bacterial antigens may penetrate through the mucosal barrier. They may be presented to dendritic cells and macrophages. Furthermore, bacterial products may stimulate the epithelium directly through a receptor-mediated process (surface Toll-like receptors, cytosolic NOD2 protein receptor). Activated antigen-presenting cells as well as the epithelium produce cytokines and chemokines that recruit and activate mucosal immune cells. The cytokines IL-12 and IL-18 may contribute to the differentiation of CD4+ lymphocytes to the T helper cells 1 phenotype (Th1). The overwhelming response leading to gut injury seems to result from an inappropriate ongoing activation of the immune system (Th1 type), which is inadequately counterregulated by a protective immunosuppressive response (TR1, Th3, Th2). The balance between pro-inflammatory cytokines (IL-12, IL-18, IFN-γ, TNF-α, IL6, IL-2, IL-1, IL-17) and anti-inflammatory cytokines (IL-4, IL-5, IL-10, TGF-β) is disturbed. Furthermore, activated T-cells are resistant against apoptosis and the inflammation maintains itself.

In sclerosing cholangitis the mechanism by which autoantibodies or abnormally activated T-cells lead to clinical expression of the disease is less well known. However, a tight association with inflammatory bowel disease is obvious (inflammatory bowel disease is present in around 60% of sclerosing cholangitis) and a similar mechanism as in inflammatory bowel disease is discussed.

Crohn's disease is a chronic inflammation that can affect any part of the gastrointestinal tract, primarily the bowel. Furthermore, it is frequently associated with systemic manifestations (skin, joints, eyes). Inflammation is proposed to result from an inappropriate immune reactivity to the bacterial flora of the intestine of individuals, who are genetically susceptible. This severe inflammation is maintained by an ongoing activation of the immune system as a consequence of an irreversible imbalance favoring a pro-inflammatory over a protective anti-inflammatory immune response (Podolsky, Inflammatory bowel disease. N Eng J Med 2002, 347:417-429). The consequence is a disease with a massive reduction of the quality of life. It often requires disabling surgery and is associated with a high mortality. Since incidence and prevalence of Crohn's disease are rising, the effect of this disorder on health spending is considerable.

All these inflammatory-associated disorders are chronic, progressive conditions of unknown origin, leading to complication induced by the inflammatory process, often requiring disabling surgery. Furthermore, in both settings the risk to develop cancer in mammal is increased and the diseases are associated with a high mortality.

“Mammal” refers to any animal classified as a mammal including humans, domestic and farm animals, and zoo, sports or pet animals, such as dogs, horses, cats, cows, monkeys, etc. Preferably the mammal is a human.

“Pro-inflammatory markers”, as used herein, refer to molecules such as cytokines, chemokines, proteins, lipids, amino acids, hormones and chemical compounds that are generated by injured tissues to signal the presence of an abnormality requiring adaptation of the functioning of the organism. The pro-inflammatory markers can be selected from the group comprising INOS, IL-R1a, IL-1, TNF-α, IL-6, IL-12, IL-17, IL-18.

Preferably pro-inflammatory markers are IFN-γ, IL-R1a and TNF-α, IL-6, IL-17.

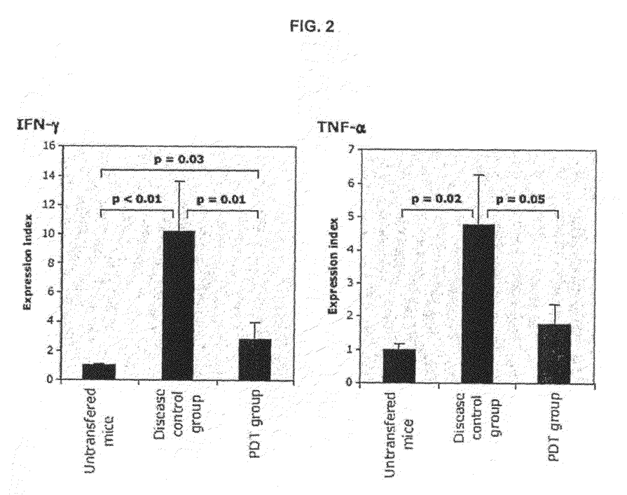

The decrease of the expression of pro-inflammatory markers in a tissue of the gastrointestinal tract according to the present invention refers, usually, to a diminution of the expression index of the expression of said pro-inflammatory markers equal or superior to 5%, preferably equal or superior to 20%, more preferably equal or superior to 40%, most preferably equal or superior to 60%, in particular equal or superior to 70% when compared to non-treated gastrointestinal tract inflamed tissues in “colonoscopy” mice, as referenced for example in FIG. 2.

Typically, the expression index of each pro-inflammatory marker of interest is calculated (normalized number of mRNA copies of each manipulated mice/normalized number of mRNA copies of the unmanipulated mice). The results are given as mean values of the expression index (unmanipulated mean mice index=1).

As can be deduced for IFN-γ and TNF-α from FIG. 2-Example 1, a decrease of the expression of pro-inflammatory marker IFN-γ (group: 10 J/cm², 15 mg/kg of δ-ALA) of 73% and a decrease of the expression of pro-inflammatory marker TNF-α (group: 10 J/cm², 15 mg/kg of S-ALA) of 63% when compared to the respective “colonoscopy” mice, i.e. mice that have been subjected to colonoscopy but not treated with PDT, can be obtained with the present invention.

Determination of the diminution of the expression index of the pro-inflammatory markers by statistical analysis such as Mann-Whitney tests are well known by those skilled in the art. Typically, a diminution of the expression index (when compared to “colonoscopy” mice) showing a p value <0.05 will be considered as significant.

It will be understood that the decrease of the expression of said pro-inflammatory markers can be assessed on, for example Polymerase Chain Reaction (PCR, RT-PCR), immunocytochemical/histochemical assays, assessing enzymatic activity, ELISA after dissection. However, any techniques that are suitable for assessing the decrease of the expression of pro-inflammatory markers can be used in the present invention.

According to the invention, the mammal is administered an amount of the medicament comprising the photosensitizing agent, or a mixture of photosensitizing agents, in one or several dosages. This in a fashion consistent with good medical practice, taking into account the nature of the inflammation being prevented or reduced, the species and medical condition of the mammal, the presence of any other drug in the subject's body, the purity and chemical form of the photosensitizing agent, the mode of administration, the rate and degree of absorption expected, and other factors known to practitioners.

The appropriate dosage form will depend on the disease, the photosensitizing agent, and the mode of administration; possibilities include tablets, capsules, lozenges, dental pastes, suppositories, inhalants, solutions, ointments and parenteral depots.

The dose as well as the choice of the photosensitizing agent will vary with the target tissue and, if administered topically or systemically, will be limited by the weight and optimal blood level of the mammal. Usually a dose sufficient to decrease the expression of pro-inflammatory markers is applied. Suitable systemic amounts per dose are typically less than 60 mg/kg of body weight, preferably less than 50 mg/kg, more preferably less than 40 mg/kg, most preferably less than 30 mg/kg, in particular less than 20 mg/kg, most particular equal or less than 15 mg/kg of body weight.

In-vitro assays will be useful for the determination of the dose of photosensitizing agent to be administered.

Depending on the photosensitizing agent and the mode of administration, an equivalent optimal systemic blood level can be established, but it is difficult to do because the photosensitizer preferably clears very rapidly. Thus, there can be a dramatic difference between the concentration of the photosensitizer in the bloodstream at the moment of injection and the concentration at the time of treatment with light.

When administered topically or systemically, the contact of the mammal with the medicament comprising the photosensitizing agent generally takes place for at least one minute, preferably under five minutes, and even more preferably from about one to two minutes. The time of contact depends on such factors as the concentration of the photosensitizing agent in the medicament, the tissue to be treated, and the particular type of medicament. After a predetermined contact time of the tissue of the gastrointestinal tract with the photosensitizing agent, the excess photosensitizing agent is preferably removed from the area of treatment.

In case of systemic administration, the photosensitizing agent is selected to have, not only rapid pharmacokinetic characteristics, but also susceptibility to rapid clearance from the body.

Following the step of administering a photosensitizing agent to a tissue of the gastrointestinal tract of a mammal with an inflammation-associated disorder of said gastrointestinal tract, the tissue is subjected to exposure with light having a wavelength that is absorbed by the photosensitizing agent. Usually a dose sufficient to decrease the expression of pro-inflammatory markers is applied.

The dose of the light exposed is typically less than 50 J/cm², preferably less than 40 J/cm², more preferably less than 30 J/cm², most preferably less than 20 J/cm², in particular equal or less than 15 J/cm², more particular equal or less than 10 J/cm², and most particular equal or less than 5 J/cm².

During the irradiation step, any light absorbed by the photosensitizing agent and that is appropriate for use with the inflamed tissue may be used, usually light from 300 to about 1200 nm, depending upon the photosensitizer and upon the depth of tissue penetration desired, preferably from 400 to about 900 nm. For general anti-inflammatory applications, red light, green light, blue light, UVA light, or even white light may be used. Light having a wavelength shorter than 400 nm is acceptable, but not preferred because of the potentially damaging effects of UVA light. Light having a wavelength longer than 700 nm is also acceptable, but not particularly preferred because of the penetration depth.

Usually, the time between administering the photosensitizing agent to the tissue of the gastrointestinal tract of a mammal with inflammation-associated disorder of said gastrointestinal tract and exposing said tissue to a light having a wavelength absorbed by said photosensitizing agent will be between 1 minute and 6 hours. Preferably, said time is 3 hours.

Exposing said tissue to a light may usually be performed using either laser diodes or light emitting diodes (LED). Any light sources (laser or non-laser) that are suitable for PDT and that are well known in the art can be used in the present invention.

The exposing time of the tissue of the gastrointestinal tract of a mammal with inflammation-associated disorder of said gastrointestinal tract to a light having a wavelength absorbed by said photosensitizing agent will, usually, be less than 600 seconds, preferably less than 500 seconds, more preferably less than 400 seconds, most preferably less than 300 seconds, in particular less than 200 seconds, more particular less than 100 seconds, and most particular less than 80 seconds, in particular equal or less than 50 seconds.

Also encompassed by the present invention is the preparation of a medicament of the invention that further comprises an immunomodulatory agent. Said immunomodulatory agent may be an immunosuppressive agent with the ability to enhance the anti-inflammatory effect on the inflamed tissue by suppressing or masking T-lymphocyte responses. This would also include agents that suppress cytokine production, down-regulate or suppress self-antigen expression, or mask the MHC antigens.

Examples of such agents include, but are not limited to, 2-amino-6-aryl-5-substituted pyrimidines; azathioprine or cyclophosphamide; bromocryptine; glutaraldehyde; antiidiotypic antibodies for MHC antigens; cyclosporin A; one or more steroids, preferably corticosteroids and glucocorticosteroids such as prednisone, methyl prednisolone, and dexamethasone; anti-interferon-gamma antibodies; anti-tumor necrosis factor-alpha antibodies; anti-tumor necrosis factor-beta antibodies; anti-interleukin-2 antibodies; anticytokine receptor antibodies such as anti-IL-2 receptor antibodies; heterologous antilymphocyte globulin; pan-T antibodies, preferably OKT-3 monoclonal antibodies; antibodies to CD4; streptokinase; streptodomase; or RNA or DNA from the host.

This immunomodulatory agent may be administered simultaneously or separately, systemically or topically. The effective amount of such agents is subject to a great deal of therapeutic discretion and depends on the amount of the photosensitizing agent present in the formulation, the type of injury, the type of immunosuppressive agent, the site of delivery, the method of administration, the scheduling of administration, other factors discussed above, and other factors known to practitioners. However, the amount of immunosuppressive agent appropriate for use with the invention is typically lower than that normally advisable for the treatment of like target tissues.

When an immunosuppressive agent is used, it may be administered by any suitable means, including parenteral and, if desired for local immunosuppressive treatment, intralesionally, i.e., topically to the target tissues. Parenteral infusions include intramuscular, intravenous, intraarterial, intraperitoneal, subcutaneous, and subconjunctival administration.

In addition to immunomodulatory agents, anti-angiogenic agents or neuroprotective agents can also be used. Exemplary neuroprotective compounds include free radical scavengers, e.g., Ebselen, Tirilazad, ganglioside, citicholine and vitamin E, GABA agonist, e.g., Clomethiazole, Ca channel antagonist, e.g., Nimodipine and Flunarizine, K channel agonist, e.g., BMS-204352, Na Channel antagonist, e.g., Fosphenyloin, and glutamate receptor antagonist, e.g., Eliprodil, Cerestat and Selfotel.

Exemplary anti-angiogenic compounds include matrix metalloproteinase inhibitor, e.g, AG3340 and marimastat, integrin antagonist, eg., EMD121974 and Vitaxin, PKC inhibitor, e.g, PKC412 and LY 333531, VEGF receptor antagonist, e.g., CEP-5214, ZD4190, SU5416 and c-p1C1 1, angiostatic steroid, e.g., squalamine and anecortave acetate, somatostatin analog, anti VEGF, e.g, NX1838 and Genentech rhMAb anti-VEGF, and other molecules such as thalidomide, IM862, angiozyme, endostatin, angiostatin, shark cartilage extracts, e.g., BeneFin and AE-941 and TNP-470.

Other agents known as increasing the efficacy of the photosenziting agent, such as for example dendrimers, insulin, immunoglobulins, avidin-biotin complexes, fluocarbonate emulsions, antibodies, ascorbate and iron, can be administered as well.

If the medicament comprises a further agent such as an immunomodulatory agent, anti-angiogenic agents, neuroprotective agents or an agent increasing the efficacy of the photosenziting agent, an effect on the doses of photosensitizing agent and the dose of light exposed might occur.

Also with in the scope of the present invention is the use of a photosensitizing agent for the preparation of a medicament for decreasing the expression of pro-inflammatory markers in a tissue of the gastrointestinal tract of a mammal having an inflammation-associated disorder of said gastrointestinal tract.

Further encompassed by the present invention is a method for reducing or preventing an inflammation-associated disorder in the gastrointestinal tract of a mammal comprising the steps of:
a) administering a photosensitizing agent to a tissue of the gastrointestinal tract of a mammal,
b) exposing said tissue of the gastrointestinal tract of a mammal to a light having a wavelength absorbed by said photosensitizing agent, wherein the expression of pro-inflammatory markers in said tissue of the gastrointestinal tract of a mammal is decreased after exposing.

Embraced by the present invention is also a method for decreasing the expression of pro-inflammatory markers in a tissue of the gastrointestinal tract of a mammal having an inflammation-associated disorder comprising the steps of:
a) administering a photosensitizing agent to a tissue of the gastrointestinal tract of a mammal,
b) exposing said tissue of the gastrointestinal tract of a mammal to a light having a wavelength absorbed by the photosensitizing agent.

This invention also concerns the use of a photosensitizing agent for the preparation of a medicament for decreasing the expression of pro-inflammatory markers in a tissue of the gastrointestinal tract of a mammal having an inflammation-associated disorder of said gastrointestinal tract.

Another concern of the present invention is the use of a photosensitizing agent for the preparation of a medicament for inactivating a grain-positive or a gram-negative bacterial cell related to an inflammation-associated disorder of the gastrointestinal tract in a tissue of the gastrointestinal tract of a mammal having an inflammation-associated disorder of said gastrointestinal tract.

Inactivation of a gram-positive or a gram-negative bacterial cell related to an inflammation-associated disorder of the gastrointestinal tract happens simultaneously, or after, as the decrease of the expression of pro-inflammatory markers after administering the photosensitizing agent and exposing the tissue to a light having a wavelength absorbed by said photosensitizing agent.

The foregoing description will be more fully understood with reference to the following Examples. Such Examples, are, however, exemplary of methods of practicing the present invention and are not intended to limit the scope of the invention.

### EXAMPLES

### Example 1

### Material and Methods

For bowel cleansing chow was taken away from mice and drinking water was replaced by Fordtran (65 g/l; Streuli & Co, Uznach, Switzerland). Mice were anesthetized by intraperitoneal injection of a solution of phospate buffer saline (PBS) containing 40% of Ketaminol 5 (50 mg/ml solution; Intervet, Zuerich, Schweiz) and 10% of Rompun (Bayer, Zuerich, Switzerland) in a dosage of 5 μl/g body weight. Endoscopy in mice was performed with two types of endoscopes:

a) A home made flexible bundle multi-fiber-mini-endoscope (length 40 cm, diameter 1.2 mm) with a standard endoscopic ocular (EPFL Lausanne, Switzerland), a home made Xenon lamp using a BULB M24N002 (Welch Allyn WA, USA), a Camera Telecam SL PDD (Storz Inc, Tuttlingen, Germany) and a home made air inflation system consisting of an low pressure air pump with electrical flow regulation, an Y adapter with lateral flush (PSFLL, Wilson Cook Bloomington Ind., USA) and a polyester shrink white tubing (052200WST Advanced Polymers, Salem NG, USA.

b) A rigid mini-endoscope Hopkins I (vision direct 0°, length 10 cm, diameter 1.9 mm, Anklin, Binningen, Switzerland) with a 9 Charrière (Ch) tube and a channel for instrumentation (Flexible biopsy forceps Ch 3, 53 cm length) coupled on the Coloview Basic equipment (light source Xenon 175 and Endovision Telecam SLB; Storz Inc, Tuttlingen, Germany) and the home made air inflation system (see above). All images were displayed on Sony color monitor (Schlieren, Switzerland) and stored via a SONY video recorder. First the colonoscopy technique was optimised in 10 wild type BALB/c mice and 10 SCID mice, which were colonoscoped repeatedly (3 times within a 2 weeks time period). Afterwards the accuracy of colonoscopy in diagnosis colitis was evaluated in BALB/c mice with sodium dextran sulfate (DSS) induced colitis and in SCID mice with colitis induced by transfer of a subpopulation of CD4+CD45RBhigh T-cells. For this purpose the mice were colonoscoped at diverse time points after onset of DSS administration or T-cell transfer and the endosocpic image was compared with the endoscopic image of normal mice and the histology after sacrificing the mice.

The BALB/c and SCID mice used in this experiment were purchased from Harlan (Netherland). Mice were used at 6 weeks of age and maintained in compliance with the Swiss Council on Animal Care Guidelines. The Veterinary Authorization delivered by the Service Veterinaire Vaudois (Lausanne) for the SCID mice colitis was 1527.

### Induction of Colitis

For DSS induced colitis, DSS in a dosage of 5% was administered with the drinking water over a time period of 7 days.

To induce CD4+ CD45RBhigh transfer colitis, T cells used for the adoptive transfer of the SCID mice were obtained from the spleens of six weeks old wild type BALB/c mice that were housed under specific pathogen-free (SPF) conditions at our animal care facility. Mice were sacrificed by cervical dislocation under anesthesia and spleen were recovered and kept in cold RPMI 1640 medium complemented with a final concentration of 2% fetal calf serum (FCS) until processing. The tissue was forced through 70 μm and 40 μm nylon meshes and washed. Spleen cells were then centrifugated and the pellet was resuspended in 5 ml of cold medium for counting. The cellular preparation was then enriched in CD4+ cells by magnetic cell sorting using CD4 (L3T4) MACS microbeads (Miltenyi Biotec, Gladbach, Germany). The enriched cells were then stained using fluorescein isothiocynate (FITC)-conjugated anti-CD4 and phyoerythrin (PE)—conjugated anti-CD45RB monoclonal antibodies (BD, Biosciences Pharmingen, San Diego, USA). CD4+CD45RB high cells were sorted by FACS, resuspended in PBS at the concentration of 10⁶ cells/ml and finally 10⁵ cells were injected intravenously under sterile conditions into 4-6 weeks old SCID mice.

### Photodynamic Therapy (PDT) in Mice

Freshly prepared delta-aminolevulinic acid (δ-ALA) was administered intragastrically after anesthesia by isofluorane inhalation. For illumination a 5 French endoscopic Huibretgse Cotton set catheter (HBSs, Wilson Cook, Bloomington Ind.) with a 2.5 cm long, radial laser diffuser (RD-20, diameter 0.95 mm, Medlight, Ecublens, Switzerland) was introduced.

Afterwards the introducer tube was pulled back by 2.5 cm, while the fiber was held in place. As light source served a dye laser (375 B, Spectra-Physics, 375B, Irvine, Calif., USA) pumped by an Argon Ion Laser (Innova 100, Coherent Inc. Santa Clara, Calif., USA). For illumination 635 nm wavelength and a power density of 100 mW/cm2 was used. After PDT mice were kept in dim light for 2 days.

### Procedures and Time Schedule

Wild type BALB/c mice were labeled and weighted, quality of life was assessed, blood samples were taken, chow and drinking water was taken away and bowel cleansing was performed with Fordtran (see above) at 0 hours (h). Oral δ-ALA was administered via gavage in the PDT groups (group 8-10) and δ-ALA only groups (group 6,7) at 5 h and colonoscopy was performed at 8 h in all groups beside the negative control group 1. At the same time illumination of the left colon was performed in the PDT groups (group 8-10) and the illumination only groups (group 3-5). The illumination time in the 5 J/cm2, 10 J/cm2 and 50 J/cm2 was 50 s, 100 s and 500 s, respectively. After the colonoscopy chow and plain drinking water were put back.

### Dose Groups

10 experimental groups of mice (n=5) were examined:
Group 1: negative control mice (unmanipulated mice)
Group 2: colonoscopy only (mock control mice)
Group 3: illumination only with 5 Joule/cm² (0 mg δ-ALA)
Group 4: illumination only with 10 Joule/cm² (0 mg δ-ALA)
Group 5: illumination only with 50 Joule/cm² (0 mg δ-ALA)
Group 6: administration of 15 mg δ-ALA only (0 Joule/cm²)
Group 7: administration of 60 mg δ-ALA only (0 Joule/cm²)
Group 8: low dose PDT with 5 Joule/cm², 15 mg δ-ALA

### DETAILED DESCRIPTION OF THE INVENTION

The present invention relates to the use of a photosensitizing agent for the preparation of a medicament for the treatment or prevention of an inflammation-associated disorder in the gastrointestinal tract of a mammal, wherein the expression of pro-inflammatory markers in a tissue of said gastrointestinal tract is decreased after administering said photosensitizing agent to said tissue and exposing said tissue to a light having a wavelength absorbed by said photosensitizing agent.

As used herein, “a” or “an” means “at least one” or “one or more.”

As used herein, “tissue” refers to a collection of similar cells and the extracellular substances surrounding them.

As used herein “gastrointestinal tract” refers to the tubular organ extending from mouth to anus and its side organs that include liver and pancreas

“Administering”, as it applies in the present invention, refers to contact of a pharmaceutical agent or composition, to the subject, preferably a mammal, most preferably a human.

A “photosensitizing agent” or “photosensitizer”, as used herein, refers to a chemical compound that, when exposed to light of a wavelength capable of being absorbed by the photosensitizing agent, absorbs light energy to result in the desired physiological effect, e.g. in the formation of reactive oxygen species which can result in the induction of apoptosis in a wide variety of cell types. A property of photosensitizing agents in general that is of particular significance in the practice of the present invention is a relative absence of toxicity to cells in the absence of the photochemical effect and the ready clearance from tissues in the absence of a target-specific interaction between particular cells and the photosensitizing agent.

Any photosensitizing agents that are suitable for PDT and that is capable of penetrating into target cells to be treated can be used in the present invention.

Group 9: low dose PDT with 10 Joule/cm², 15 mg δ-ALA
Group 10: high dose PDT with 50 Joule/cm², 60 mg δ-ALA

### Parameters Investigated

Mice were weight from 0 h to 74 h and body weight loss was defined by percentage of weight loss from baseline bodyweight. For assessment of “Quality of Life” changes in movements and texture of the fur were closely monitors. Furthermore, signs of photosensitivity were noticed. Blood was collected on anesthetized mice by the retro-orbital punction technique at 0 and 74 h. Blood was collected in sample tubes containing heparin and blood formula were obtained using an automated Coulter Ac Tdiff hematology analyzer.

At 74 h mice were killed, the macroscopic aspect of the colon was assessed, the colons were removed through a midline incision and the illuminated part of the colon (2 cm) was collected and splitted in 3 portions: One third of the colon was used for histological analysis. Therefore, the colon harvested from the sacrificed mice was embedded in an embedding medium (Tissue-Tek, OCT, Miles, Clarkston, USA), frozen in liquid nitrogen-cooled isopentane and stored at −20° C. Frozen sections (10 μm) were obtained using a Leica Cryostat model CM 1800 apparatus and mounted on SuperFrostPlus® microscope slides (Menzel-Glase, Braunschweig, Germany). Sections were then submitted to standard hematoxylin/eosin coloration, dehydrated and mounted in glycerol. Sections were observed using an Axioplan microscope (Carl Zeiss, Feldbach, Switzerland).

In another third of the frozen tissue anti-Mac-1 immunostaining was performed. Mac-1 is expressed by macrophages and neutrophils. For this purpose the frozen sections were washed in PBS and blocked for 30 minutes with a PBS solution containg 2% FCS (PBS-S) and 5% mouse serum. This was followed by incubation with a fluorescein isothiocynate (FITC)-conjugated anti-CD 11b (Mac-1) monoclonal antibody (BD Biosciences Pharmingen, San Diego, USA) diluted 1:20 in PBS-S for 2 hours in the dark, at room temperature. After successive PBS-S washings, section were mounted in Vectashield (Vector Laboratories, Burlingame, Calif., USA) and observed using an Axioplan microscope (Carl Zeiss, Feldbach, Switzerland).

The third portion was used for measuring the expression of the molecules involved in immune and inflammatory phenomenons in the colonic mucosa (cytokines, chemokines). For RNA extraction this portion was conserved in RNA-later solution (Ambion Inc. Austin, USA) until processing. RNA was extracted from the tissues using a Rneasy Mini kit (Qiagen, Hombrechtikon, Switzerland). RNA samples were submitted to a second or third DNAse treatment in order to get rid of all traces of genomic DNA. Quality of the RNA samples was tested on agarose gels and absence of genomic DNA was tested by PCR using primers specific for a house keeping gene, glyceraldehydes-3-phosphate-dehydrogenase (GAPDH). RNA preparations were then submitted to reverse transcription using the ThermoScript™ RT-PCR system (Invitrogen, Basel, Switzerland) using an Oligo-dT as primer. Quantification of reverse transcripted messenger RNA for GAPDH, the inducible nitric oxide synthetase (iNOS), interferon-gamma (IFN-γ), interleukin-1 receptor antagonist (IL1-Ra), tumor necrosis factor alpha (TNF-α), interleukin 6 (IL-6), tumor growth factor beta (TGF-13) and interleukin10 (IL-10) was performed by quantitative real-time PCR in a BioRad Real-time PCR iCycler using specific pairs of primers and the green fluorescence dye SYBR—Green®. GAPDH was used to normalize quantifications within each individual sample (normalization of the number of mRNA copies for the gene of interest/1 million mRNA copies of the GAPDH gene of the investigated mouse). Expression of each gene of interest was finally calculated as an expression index (normalized number of mRNA copies of each manipulated mice/normalized number of mRNA copies of the unmanipulated mice) and the results are given as mean values of the expression index (unmanipulated mean mice index=1). Furthermore one kidney and a part of the liver were collected for pathological analysis to exclude hepato- or nephrotoxicity.

### Feasibility and Accuracy of Colonoscopy

Colonoscopy could safely be performed in normal BALB/c mice and SCID mice. The mouse colon could be intubated up to the right flexure. The length of the accessible mouse colon was approximately 4 cm with the rectum comprised. In DSS induced colitis a correlation between colitis and weight loss was observed, whereas in SCID mice with colitis after CD4+CD45RB^{high} T-cell transfer, endoscopic signs of colitis developed earlier as weight loss and the correlation between endoscopic signs of colitis and weight loss was less good. The colitis in DSS mice was segmental, the rectum was always spared, erythema, ulcerations, changes of the normal vascular pattern could be observed. Based on these data in DSS and SCID mice Applicants modified the endoscopic severity index of Wirtz et al (J of Immunology 2002) by adding erythema as a fifth parameter. Furthermore, since the length of the accessible mouse colon was only about 4 cm, they changed their scoring system concerning the length. This modified endoscopic severity index of colitis allows us to select mice with the same severity index for randomisation in different treatment arms, to monitor the colitis activity in individual SCID mice at different time points after treatment and to compare colitis activity in the different experimental groups.

### Safety of Colonoscopy and <<Low Dose>> PDT

<<High dose>> PDT induced a 7% loss of body weight (mean loss 1.39 gram+SEM 0.23 g; p=0.0159). Neither with <<low dose>> PDT, nor with colonoscopy nor any other experimental condition a significant change in body weight was observed.

After <<high dose>> PDT a loss of quality of life was observed. The fur of the mice became bristled, the mice moved less and they took mainly a coved posture. In all other experimental groups the exterior aspect of mice remained unchanged over the observation period of 74 h. Furthermore, no signs of skin phototoxicity were observed.

No significant changes in the blood formula were seen in any of the experimental conditions. Not only the illuminated part, but also the whole colon and the small intestine showed dilatation and edema after <<high dose>> PDT. After <<low dose>> PDT, after colonoscopy and in all other experimental groups the colon appeared macroscopically normal at 74 hours. The macroscopic changes in the <<high dose>> PDT group corresponded histologically with a loss of the villous structures, erosions, an increase in cellular infiltration in the lamina propria and a reduced muscular layer. In all other experimental groups no microscopic changes were observed and the colon mucosa appeared as in the negative control group. By anti-MAC immunostaining of frozen tissue sections an increase of inflammatory cells was observed, compared to naïve mice (data not shown). In contrast <<low dose>> PDT did not lead to an increase in inflammatory cells.

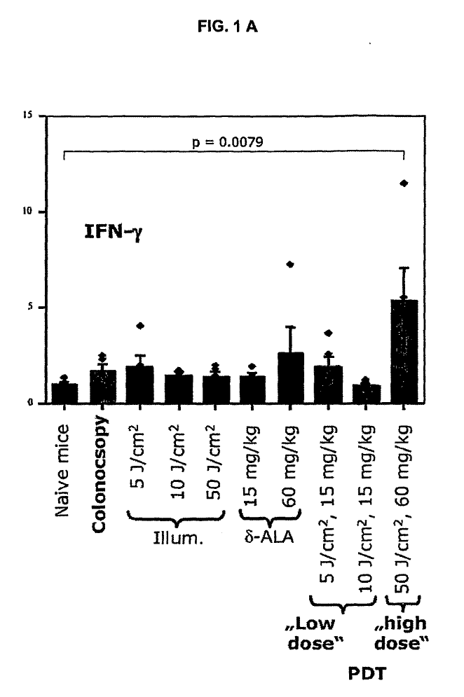

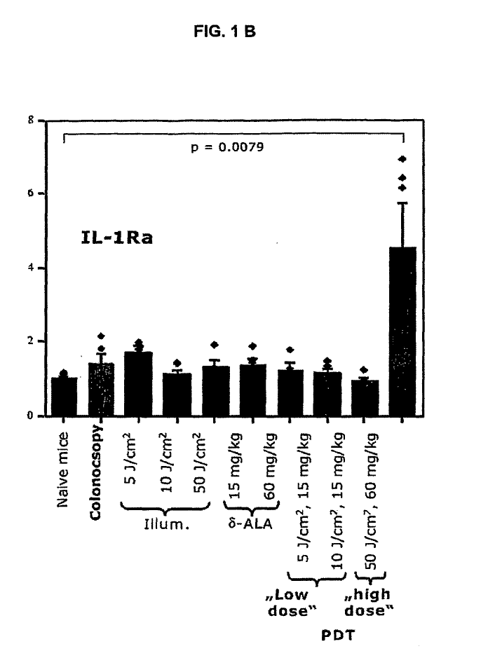

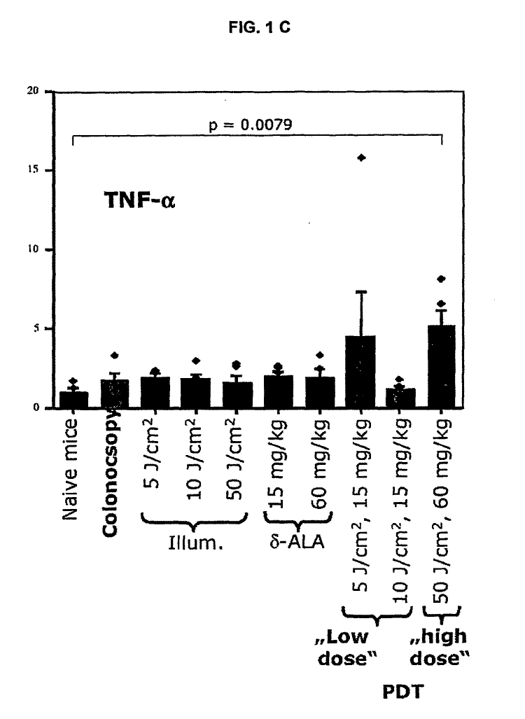

The expression indices of the pro-inflammatory markers iNOS (27.78±10.47; p=3.0317), IFN-γ (5.36±1.67; p=0.0079), TNF-α (5.19±0.93; p=0.0079) and IL-1 Ra (4.54±1.21; p=0079) were only increased in the <<high dose>> PDT group (FIGS. 1A, B and C). No increase in pro-inflammatory cytokines was observed in the <<low dose>> PDT groups or after colonoscopy. The values for colonoscopy and the <<low dose>> PDT groups with 5J/cm2, 15 mg/kg δ-ALA and 15 mg/kg δ-ALA, 10 J/cm2 were as followed: IFN-γ (1.73±0.29; 1.92±0.50; 0.93±0.09, respectively), IL-1Ra (1.38±0.27; 1.14±0.11; 0.94±0.08, respectively) and TNF α (1.80±0.40; 4.49±2.86; 1.16±0.21, respectively).

### Results:

Colitis developed very late in this example (after 7 weeks) and only 55% of transferred mice, which we were able to inspect by colonoscopy developed colitis (see above).

Unfortunately the flexible endoscope was damaged during the SCID mice experiments. Therefore the majority of transferred mice (n=42) could only be monitored clinically, and as described by Wirtz et al (J Immunol 2002) no clinical reliable sign correlates nicely with colitis in mice. In the 4 mice, which developed signs of severe disease, PDT was able to cure 2 mice and improve clinical signs in one mouse, respectively. Only one mouse (25%) did not show a clinical response.

In the mice, which applicants were able to follow-up, the endoscopic severity index improved significantly after PDT (see experimental setting above). Furthermore, the expression indices of the pro-inflammatory markers IFN-γ and TNF-α, decreased significantly after “low dose” PDT and were significantly lower than in the disease control group (FIG. 2). Weight loss and loss of “Quality of Life” did neither correlate with the endoscopic severity score, nor with cytokine expression.

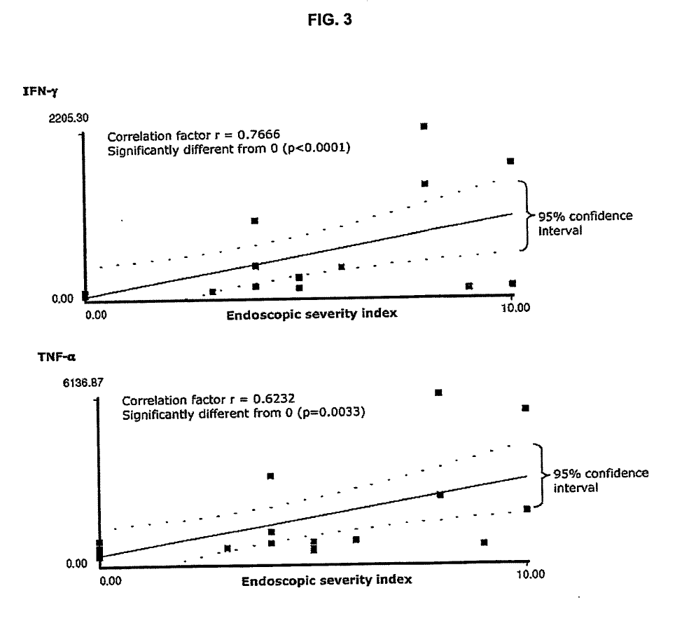

In contrast the endoscopic severity index correlated nicely with cytokine expression (see FIG. 3).

No signs of photosensitivity or damage of the mucosa was observed in any of the animals.

As can be deduced from FIG. 2 of Example 1, the results show a decrease of the expression of pro-inflammatory marker IFN-γ (group: 10 J/cm², 15 mg/kg of δ-ALA) of 73% and a decrease of the expression of pro-inflammatory marker TNF-α (group: 10 J/cm², 15 mg/kg of δ-ALA) of 63% when compared to the respective “colonoscopy” mice, i.e. mice that have been subjected to colonoscopy but neither treated with a photosensitizing agent nor exposed to a light exposure.

### Example 2

### Evaluation of the effect of <<Low Dose>> PDT on Healing of Colitis in a SCID Mouse Model, after Inducing a T_{H}1-Mediated Crohn-like Colitis after Transfer of Subpopulations of CD4+CD45RB^{high} T-Cells

### Induction of Colitis

Since in example 1 only 55% of the mice developed colitis, Applicants have injected 4×10⁵ cells CD4+CD45RB^{high} intravenously into 9.5 weeks old SCID mice in the following second set of SCID mice.

### Photodynamic Therapy (PDT) in Mice

Evolution of colitis in SCID mice was monitored every week by colonoscopic investigation after induction. Mice with moderately inflamed colons [endoscopic index of colitis severity (EICS) index 4-8] or marked inflamed colons (EICS 9-13) were randomly assigned to either a <<low dose>> PDT group or no treatment group (disease control group). Unmanipulated age-matched SCID mice served as negative controls.

PDT was performed with a photosensitizing agent (δ-ALA) dose of 15 mg/kg, administered 3 hours before the illumination with the energy dose of 10 J/cm² per gastrointestinal tract tissue.

The illumination time was 100 s, the wavelength 635 nm.

After completing the experiments with 15 mg/kg δ-ALA and 10 J/cm², the Applicants evaluated whether an energy illumination energy of 20 or 2 J/cm² would provide an equivalent or better effect on colitis. Furthermore, the Applicants sought to determine whether PDT treatment could be repeated at short term and whether this re-treatment would have a beneficial effect on colitis too. To this end, the Applicants performed another set of experiment with marked inflamed mice in which half of the PDT treated mice (15 mg/kg S-ALA and 10 J/cm²) were subjected to a second identical low dose PDT treatment at the time of the re-apparition of colitis symptoms, namely one week after the first PDT treatment.

### PDT Treatment Follow-Up

Mice were monitored by colonoscopy at day 3 and weeks 1, 2, 3 and 4 post PDT treatment. In addition to parameters investigated in example 1, CD4 immunostaining and apoptosis detection were performed.

For anti-CD4 immunostaining, frozen sections were extensively dried, fixed with 100% acetone at 4° C. and rehydrated in PBS. Sections were then blocked for 20 minutes with a PBS solution containing 0.1% BSA (Sigma-Aldrich Inc., St-Louis, USA) and 0.5% NMS. CD4 detection was performed by incubation of the sections for 1 hour with a cell culture supernatant originating from an hybridoma secreting rat anti-mouse CD4 (clone H129.19). After washing with PBS, an Alexa Fluor 488-conjugated goat anti-rat IgG at a final concentration of 5 ng/ml in PBS 0.1% BSA, 1% NMS was used as secondary antibody and incubated for 45 minutes before two last PBS washings. All incubations were done at room temperature unless specified otherwise.

For Annexin-V mediated detection of apoptosis mice were sacrificed 4 or 20 hours after low dose PDT later. Untreated inflamed mice were also sacrificed and were considered as the time zero reference. In order to extract cells from the colonic mucosa, treated portions of the colons, or equivalent portions in untreated mice, were collected, cut into small pieces and individually incubated for 20 minutes at room temperature under constant agitation in a PBS solution containing 5 mM EDTA. After centrifugation, colons samples were resuspended in a RPMI 1640 culture medium (Gibco Invitrogen, Basel, Switzerland) containing 2% FCS (Biological Industries) and 0.5 mg/ml of collagenase N (Sigma-Aldrich Inc.) and were incubated for 30 minutes at 37° C. under constant agitation. After another centrifugation, digested tissues were successively forced through 70 and 40 μm nylon cell strainer (Becton Dickinson). Individualized cells were then applied on a Ficoll-Paque™ Plus (Amersham Biosciences, Uppsala, Sweden) gradient and mononuclear cells were recovered after centrifugation. These later cells were then first stained by a 20 minutes incubation at 4° C. with a phycoerythrin-conjugated anti-mouse CD4 monoclonal antibody (clone 129.19, Becton Dickinson) diluted 1/200 in RPMI culture medium containing 2% FCS. After one washing step, cells were then stained with FITC-conjugated Annexin V (Becton Dickinson) according to the manufacturer protocol. Cells were then analyzed through a FACscan flowcytometer (Becton Dickinson), once in the absence of propidium iodide (PI) and once in the presence of PI in order to identify population of viable cells. The percentage of Annexin-V⁺ cells within the CD4⁺ population was finally calculated based on a forward and side scatter gated population consisting of viable cells (>98% of PI negative cells) and containing the most percentage of CD4⁺ cells (>80%).

### Statistical Analysis

All statistical analyses were performed using the Prism version 4.0c software from GraphPad Software (San Diego, Calif.). The unpaired two-tail Mann-Whitney test was usually applied, unless the numbers of mice in test groups were too small to perform this test (<5 mice in both test groups) and the unpaired two-tail t test was thus applied. Significance limit was set at a 2-tailed P value≦0.05.

### Results:

### Low Dose PDT Treats Colitis Symptoms in Marked Inflamed Mice

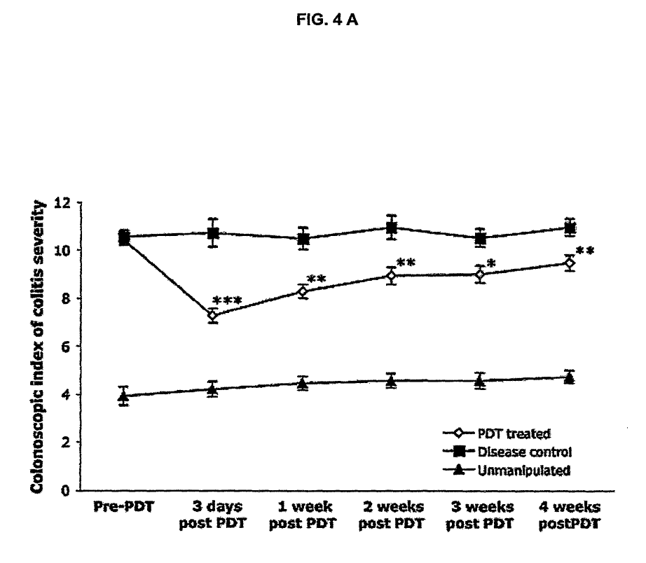

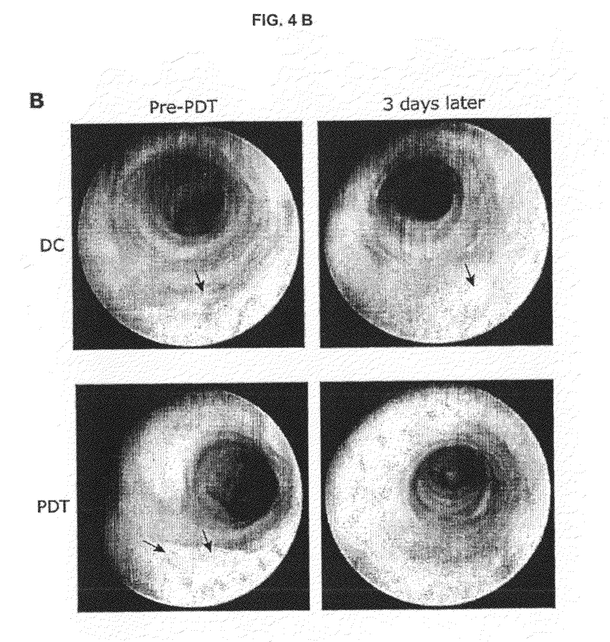

Low dose PDT (15 mg/kg aδ-ALA, 10 J/cm²) improved marked inflamed colitis (mean EICS of 10.4±0.2) already 3 days after PDT (EICS of 7.3±0.3) compared to the disease control group (10.7±0.6; P<0.0001, FIG. 4A, endoscopic image 4B). Although the EICS of PDT-treated mice slightly raised afterwards, it remained significantly lower than the one of the disease control group mice up to 4 weeks after PDT (FIG. 4A).

Histological analysis (data not shown) correlated with the EICS. The histological score of inflammation significantly dropped from 7.7±0.6 for DC mice to 4.6±0.8 3 days after PDT (P=0.0250, FIG. 4C), Histology revealed a normal mucosa with low cellular infiltration in the illuminated portion of colons of PDT-treated mice and in the colons of unmanipulated mice. In contrast the non-illuminated portion of colons of PDT-treated mice and the colons of the disease control group displayed a hypertrophied mucosa with high cellular infiltration. EICS correlated with standard indices of inflammation like reduction of colon length (correlation coefficient R=0.58; p<0.0001), increase in colon weight (R=0.55, p<10002) and mRNA expression indices for the IL-17 and IL-6 cytokines (FIG. 4C).

### Low Dose PDT Beneficial Effect on Colitis Symptoms is Delayed in Moderately Inflamed Mice

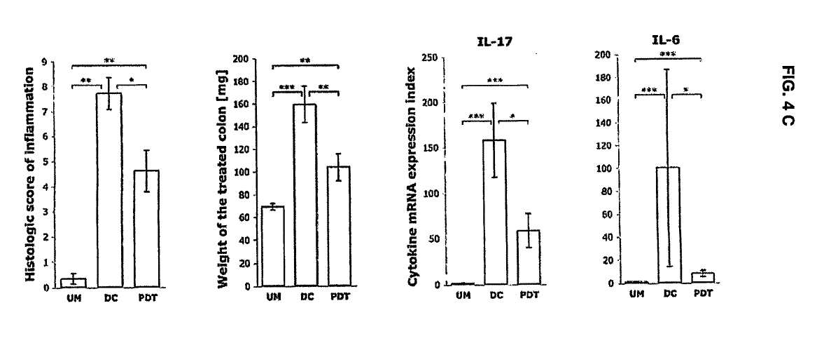

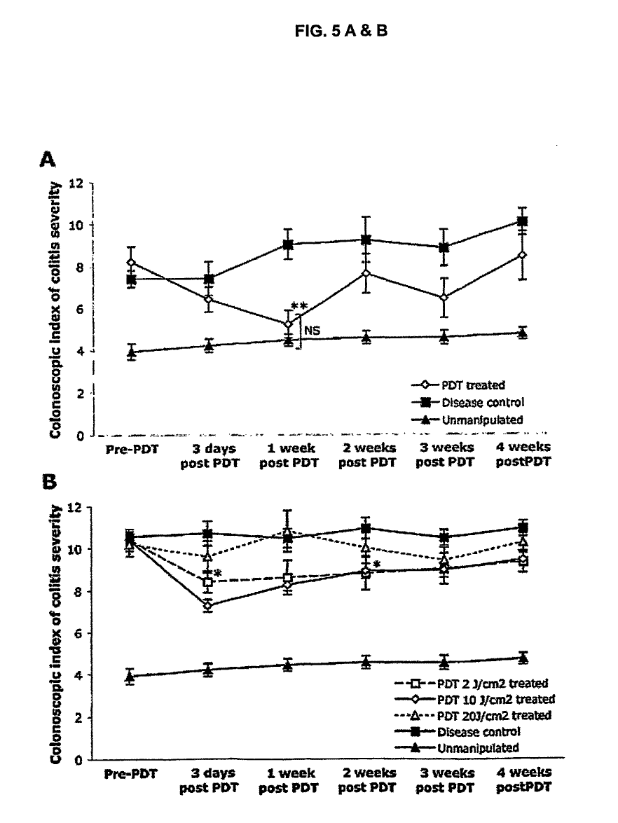

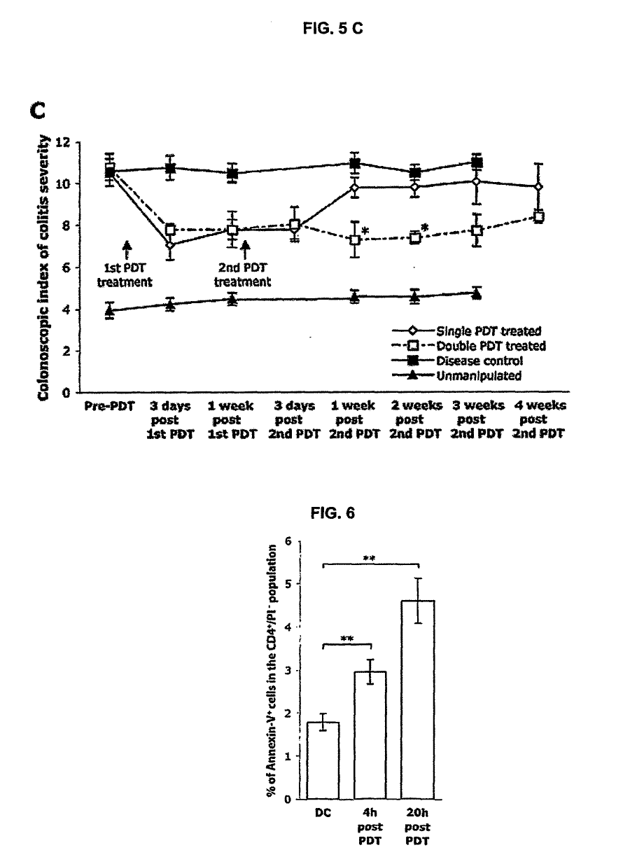

In moderately active colitis (mean EICS of 7.8±0.4) the PDT-induced effect was observed later, namely 1 week after PDT compared to the disease control group (EICS of 5.2 f 0.7 and 9.0±0.7 respectively, P=0.0079, FIG. 5A). Interestingly, at this one-week time point, EICS of PDT-treated mice did not differ from the one of age-matched unmanipulated mice (4.4±0.3, P=0.3381). Then, as already observed for marked inflamed mice, inflammation increased again up to week 4 post PDT. Since week 2, EICS of PDT-treated mice appeared no more significantly different from the one of the disease control group and became again significantly different from the one of the unmanipulated group.

### Dose Dependent Effect of PDT Regimens on Colitis Symptoms

The PDT energy dose of 20 J/cm² did not induce any significant beneficial effect on the colitis at any time point when compared to the disease control group (FIG. 5B). In contrast, the lower PDT dose regimen (2 J/cm²) induced a significant improvement of the EICS of treated mice 3 days after PDT treatment when compared to the disease control group (EICS of 8.4±0.5 and 10.7±0.6 respectively, P=0.0271, FIG. 5B). This improvement was not as strong as the one obtained at day 3 with the PDT regimen of 15 mg/kg δ-ALA and 10 J/cm².

PDT Treatment could be Efficiently Repeated and Permits to Delay the Reappearance of Colitis Symptoms in Marked Inflamed Mice

As already mentioned infra, it could be observed in the previous experiments performed with marked inflamed mice that inflammation started to slowly raise up again 1 week after PDT treatment (FIG. 4). The second PDT treatment significantly delayed the re-appearance of inflammation when compared to mice subjected to one single PDT treatment (EICS respectively and P values at the mentioned time points after second PDT treatment: 1 week, 7.3±0.9 and 9.8±0.5, P=0.0433; 2 weeks, 7.33±0.3 and 9.8±0.6, P=0.0123; FIG. 5C). This demonstrates that a second low dose PDT treatment could be fully considered, even soon after another PDT session, since it prolongs the beneficial effect on the colitis activity.

### Low Dose Pdt Causes the Disappearance of Cd4⁺ Lymphocytes in the Treated Colonic Mucosa

Having demonstrated that low dose PDT had a real therapeutic potential inducing the rapid amelioration of colitis symptoms, The Applicants sought to find out what could be the possible mechanisms of action of low dose PDT leading to this healing. As depicted in FIG. 4A, low dose PDT treatment induced an obvious diminution in the number of CD4⁺ cells present in the treated colonic mucosa when compared to DC mice. Interestingly, some CD4⁺ cells still appeared to be present in the mucosa of PDT-treated mice, in comparison to unmanipulated mice which are totally devoid of these cells. These remaining CD4⁺ cells are likely to be responsible for the residual signs of inflammation that could be observed 3 days after PDT treatment (FIG. 4).

Furthermore, the Applicants could observed that the percentage of Annexin V⁺ cells within the CD4⁺ population significantly increased at 4 and 20 hours after PDT treatment when compared to untreated DC mice (percentage of Annexin V⁺ cells in the CD4⁺/PI⁻ population at mentioned time points after PDT treatment and P values of comparison with the disease control group, time zero reference: 0 hour, 1.8±0.2%; 4 hours, 3.0±0.3%, P=0.0043; 20 hours, 4.6±0.5%, P=0.0012; FIG. 6B). Low dose PDT implemented in the colon of inflamed mice is thus able to trigger CD4⁺ cells apoptosis that are present in the inflamed colonic mucosa. This PDT-induced T cell apoptosis is likely partly responsible for the diminution in the number of pathogenic CD4⁺ cells observed in the colonic mucosa 3 days post PDT treatment (FIG. 6) and, consequently, for the beneficial effect of PDT-treatment on colitis.

1. Use of a photosensitizing agent for the preparation of a medicament for the treatment or prevention of an inflammation-associated disorder in the gastrointestinal tract of a mammal, wherein the expression of pro-inflammatory markers in a tissue of said gastrointestinal tract is decreased after administering said photosensitizing agent to said tissue and exposing said tissue to an endoluminal light application having a wavelength not longer than 700 nm absorbed by said photosensitizing agent.

2. Use according to claim 1, wherein the dose of said photosensitizing agent is less than 60 mg/kg of body weight.

3. Use according to claim 1, wherein the dose of said light exposed is less than 50 J/cm².

4. Use according to claim 1, wherein the time between administering said photosensitizing agent to said tissue and exposing said tissue to a light having a wavelength absorbed by said photosensitizing agent is between 1 and 6 hours.

5. Use according to claim 1, wherein the inflammation-associated disorder in the gastrointestinal tract is selected from the group comprising Crohn's disease, inflammatory bowel disease, microscopic colitis, sclerosing cholangiopathy, sarcoidosis, sprue, Whipple's disease, microscopic lymphocytic colitis, microscopic collagenous colitis, radiation colitis, AIDS manifestation in the gastrointestinal tract, eosiniophile gastroenteritis or esophagitis.

6. Use according to claim 1, wherein the photosensitizing agent is selected from the group comprising porphyrins, 5-aminolevulinic acid, benzoporphyrin-derivative mono acid-A, chlorins, purpurins, pheophorbides, pyropheophorbides, pheophytins, phorbins, phtalocyanines, naphthalocyanines, phenothiazine, methylene blue, texaphyrins, porphycenes, sapphyrins, synthetic dyes, hypericin.

7. Use according to claim 1, wherein the pro-inflammatory markers are selected from the group comprising INOS, IFN-γ, IL-R1a, IL-1, TNF-α, IL-6, IL-12, IL-17, L-18.

8. Use according to claim 1, wherein the medicament further comprises an immunomodulatory agent.

9. Use of a photosensitizing agent for the preparation of a medicament for decreasing the expression of pro-inflammatory markers in a tissue of the gastrointestinal tract of a mammal having an inflammation-associated disorder of said gastrointestinal tract.

10. A method for reducing or preventing an inflammation-associated disorder in the gastrointestinal tract of a mammal comprising:
a) administering a photosenzitizing agent to a tissue of the gastrointestinal tract of a mammal,
b) exposing said tissue of the gastrointestinal tract of a mammal to a light having a wavelength absorbed by said photosensitizing agent, wherein the expression of pro-inflammatory markers in said tissue of the gastrointestinal tract of a mammal is decreased after exposing.

11. A method for decreasing the expression of pro-inflammatory markers in a tissue of the gastrointestinal tract of a mammal having an inflammation-associated disorder comprising:
a) administering a photosensitizing agent to a tissue of the gastrointestinal tract of a mammal,
b) exposing said tissue of the gastrointestinal tract of a mammal to a light having a wavelength absorbed by the photosensitizing agent.
